(19) United States
(12) Patent Application Publication
(10) Pub. No.: US 2007/0161095 A1
(43) Pub. Date: Jul. 12, 2007

(54) Biomass Fuel Synthesis Methods for Increased Energy Efficiency

(76) Inventor: Michael H. Gurin, Glenview, IL (US)

Correspondence Address: MICHAEL H. GURIN, 4132 COVE LANE, UNIT A, GLENVIEW, IL, 60025, US

(21) Appl. No.: 11/691,070

(22) Filed: Mar. 26, 2007

Related U.S. Application Data
(63) Continuation-in-part of application No. 11/306,911, filed on Jan. 16, 2006.
(63) Continuation-in-part of application No. 11/309,025, filed on Jun. 12, 2006.
(60) Provisional application No. 60/767,403, filed on Mar. 25, 2006.
(60) Provisional application No. 60/593,485, filed on Jan. 18, 2005.
(60) Provisional application No. 60/595,167, filed on Jun. 13, 2005.

(51) Int. Cl. C12P 7/64; C12P 7/06

(52) U.S. Cl. 435/134; 435/161

(57) ABSTRACT

A high efficiency method for synthesizing biomass fuels leveraging the synergistic impact of ionic liquids on both the significant gains in pretreatment of biomass and the utilization of the combination of ionic liquids and carbon dioxide under supercritical conditions for energy generation is provided. The strategic use of heat exchangers, preferably microchannel heat exchangers and microchannel reactors further increase the efficiency and performance of the system by extensive heat recovery and the direct utilization of the biomass solution as the working fluid of a thermodynamic cycle.

### CROSS-REFERENCE TO RELATED APPLICATIONS

This application claims priority to U.S. Provisional Patent Application Ser. No. 60/767,403, filed on Mar. 25, 2006, for “Biomass Fuel Synthesis Methods for Increased Energy Efficiency.” This application is also a continuation-in-part of U.S. patent application Ser. No. 11/306,911, filed on Jan. 16, 2006, which claims priority to U.S. Provisional Patent Application Ser. No. 60/593,485, filed on Jan. 18, 2005, for “High Efficiency Absorption Heat Pump and Methods of Use.” This application is also a continuation-in-part of U.S. patent application Ser. No. 11/309,025, filed on Jun. 12, 2006, which claims priority to U.S. Provisional Patent Application Ser. No. 60/595,167, filed on Jun. 13, 2005, for “Nano-Ionic Liquids and Methods of Use.” Each of these applications is incorporated herein by reference in its entirety.

### FIELD OF THE INVENTION

The invention is directed to the synthesis of biomass fuels utilizing supercritical fluids and ionic liquids and a range of supercritical process methods that enable high-energy efficiency conversion and transformation to alternative fuels including biofuels.

### BACKGROUND

Cellulose ethanol requires more advanced pretreatment methods because the sugar carbon components—cellulose and hemicellulose—are much more difficult to hydrolyze economically into fermentable sugars. After the cellulose and hemicellulose have been saccharified, the remainder of the ethanol production process is similar to grain-ethanol. Hydrolysis (saccharification) breaks down the hydrogen bonds in the hemicellulose and cellulose fractions into their sugar components: pentoses and hexoses. These sugars can then be fermented into ethanol.

Ionic liquids “IL” can affect dissolution of celluloses from a variety of sources including plants, silk fibroin, and wool with no degradation of the solutes. In the case of cellulose, the solvation mechanism is proposed to involve the interaction of the IL chloride ions, which are non-hydrated and in a concentration of approximately 20 weight %.

High-resolution 13C NMR studies of cellulose and cellulose oligomers dissolved in the ionic liquid (IL) 1-butyl-3-methylimidazolium chloride ([C4mim]Cl) show that the b-(1A4)-linked glucose oligomers are disordered in this medium and have a conformational behavior which parallels the one observed in water, and thus, reveal that the polymer is disordered IL solution as well.

The chloride ions present in [C4mim]Cl solutions, which are non-hydrated and in a concentration of approximately 20 wt %, effectively break the extensive hydrogen bonding network of the polysaccharide by interacting with its hydroxyl groups, thereby promoting cellulose dissolution with no apparent degradation of the glycosidic bonds. In order to better understand how [C4mim]Cl effects cellulose dissolution, the conformational behavior of the polysaccharide upon solvation by this IL needs to be investigated.

ILs are capable of dissolving carbohydrates ranging from simple sugars to polysaccharides. Some of the best results in this regard have been obtained with [C4mim]Cl. As recently shown, the nonhydrated chloride ions present in solutions of this IL solvate carbohydrates by forming hydrogen bonds with their hydroxyl groups. For example, cellulose solutions in concentrations of up to 25 wt % can be obtained with [C4mim]Cl.

Ionic liquids including 1-butyl-3-methylimidazolium chloride (“Ionic liquids as green solvents: Engineering new bio-based materials” by Richard P. Swatloski, John D. Holbrey, Scott K. Spear, and Robin D. Rogers, Department of Chemistry and Center for Green Manufacturing, The University of Alabama, Tuscaloosa, Ala. 35487); “Use of ionic liquids in the study of fruit ripening by high-resolution CNMR spectroscopy: ‘green’ solvents meet green bananas” by Diego A. Fort et al. received (in Columbia, Mo., USA) 23 Oct. 2005, first published as an Advance Article on the web 19 Jan. 2006 that banana pulps at any ripening stage can be completely dissolved in solvent systems based on the ionic liquid (IL) 1-n-butyl-3-methylimidazolium chloride ([C4mim]Cl).

Jose Iborra of the University of Murcia in Spain and co-workers further used a combination of supercritical carbon dioxide and ionic liquids to help an enzyme transform some organic molecules. Unfortunately, enzymes typically don't work well in carbon dioxide. So Iborra's group devised a two-phase reactor in which the organic starting materials are dissolved in supercritical carbon dioxide and passed through a chamber containing a yeast enzyme dissolved in an ionic liquid.

A wide range of pretreatment processes exist including the following:

U.S. Pat. No. 6,267,309 for “Municipal solid waste processing facility and commercial ethanol production process” to Chieffalo, et al. utilizes concentrated sulfuric acid as a means of solubilizing the cellulose within a pretreatment step.

U.S. Pat. No. 5,135,861 for “Method for producing ethanol from biomass” to Pavilon utilizes a mixture of biomass and water that subsequently produces carbon dioxide “CO2” as a byproduct. The carbon dioxide byproduct from the initial fermentation product subsequently aids the catalytic hydrolysis conversion of the biomass. The '861 patent furthermore limits the operating pressure well below the supercritical pressure of CO2.

United States Patent Application No. 20020164731 for “Process for the simultaneous production of xylitol and ethanol” to Eroma, Olli-Pekka et al. utilizes traditional pretreatment processes for biomass hydrolysates selected from the group consisting of direct acid hydrolysis of said biomass, enzymatic prehydrolysate obtained by prehydrolysis of said biomass with steam or acetic acid, acid hydrolysis of prehydrolysate obtained by prehydrolysis of said biomass with steam or acetic acid, autohydrolysis using water or steam, and a sulphite pulping process.

U.S. Pat. No. 5,711,817 for “Method for the continuous conversion of cellulosic material to sugar” to Titmas utilizes gravity as a means of increasing the cellulosic material liquid stream (a.k.a. biomass slurry) pressure in order to increase the thermodynamic efficiency.

United States Patent Application No. 20050069998 for “Procedure for the production of ethanol from lignocellulosic biomass using a new heat-tolerant yeast” to Ballesteros Perdices, Ignacio et al. utilizes traditional steam explosion pretreatment in with the combination of cellulase (CELLUCLAST 1.5L, from NOVO-NORDISK) and beta.glucosidase (NOVOZYME 188 from the NOVO-NORDISK) and culture of the heat-tolerant yeast Kluyveromyces marxianus CECT 10875.

U.S. Pat. No. 6,090,595 for “Pretreatment process for conversion of cellulose to fuel ethanol” to Foody, et al. utilizes an improved pretreatment by varying the feedstock with a ratio of arabinoxylan to total nonstarch polysaccharides (AX/NSP) of greater than about 0.39, or a selectively bred feedstock on the basis of an increased ratio of AX/NSP over a starting feedstock material, and reacting at conditions that disrupt the fiber structure and hydrolyze a portion of the cellulose and hemicellulose. The pretreatment in every other manner remains traditional wherein the pretreatment is carried out with a steam explosion or extrusion device during the reaction step.

United States Patent Application No. 20040231661 for “Method of processing lignocellulosic feedstock for enhanced xylose and ethanol production” to Robert Griffin et al. utilizes multiple steps beginning with leaching a mechanically disrupted lignocellulosic feedstock prior to any pretreatment of the feedstock and ending with reacting said acidified feedstock under conditions which disrupt fiber structure and hydrolyse a portion of hemicellulose and cellulose of said acidified feedstock, to produce a composition comprising xylose and a pretreated feedstock. Reacting acidified feedstock under conditions that disrupt the fiber structure are contemplated in the method of the '661 application and may be performed according to any method known in the art, for example, but not limited to pretreatment by steam explosion.

U.S. Pat. No. 6,824,599 for “Dissolution and processing of cellulose using ionic liquids” to Swatloski et al. utilizes a method for dissolving cellulose that comprises admixing cellulose with a molten ionic liquid that is molten at a temperature of about −10 to about 100 degree Celsius and in the substantial absence of water or a nitrogen containing base to form an admixture. Furthermore, the '599 patent discloses a method for dissolving cellulose that comprises the steps of: (a) admixing cellulose with an ionic liquid comprised of cations and anions in the substantial absence of water to form an admixture, wherein said ionic liquid is molten at a temperature of about −44 degree C. to about 120 degree C. wherein said cations contain a single five-membered ring that is free of fusion to other ring structures and said anions are halogen, pseudohalogen, or C.sub.1-C.sub.6 carboxylate; (b) irradiating said admixture with microwave radiation to assist in dissolution. The phrases “substantial absence” and “substantially free” are used synonymously to mean that less than about 5 weight percent water is present, for example. More preferably, less than about one percent water is present in the composition. The same meaning is intended regarding the presence of a nitrogen-containing base. Cellulose can be dissolved without derivitization in high concentration in ionic liquids by heating to about 100 degree C., by heating to about 80 degree C. in an ultrasonic bath, and most effectively by using microwave heating of the samples using a domestic microwave oven. Using a microwave heater, it is preferred to heat the admixture of hydrophilic ionic liquid and cellulose to a temperature of about 100 degree to about 150 degree C. Microwave heating significantly enhances the dissolution of cellulose in ionic liquids. Microwave-induced dissolution of cellulose in ionic liquids is a very quick process so that decay of the degree of polymerization is reduced. Being a relatively fast process, dissolution is energy efficient. Heating of the samples is usually required to enable dissolution. The effect of that heating may be to permit the ionic liquid solvent to penetrate into the fiber wall, which enables breaking of the fiber and microfibril structure and competitive hydrogen-bonding with encapsulated water. Ionic liquids are very efficiently heated under microwave conditions. Thus, highly localized temperatures can be obtained that promote dissolution of cellulose by disrupting the strong, water mediated hydrogen-bonding of the natural polymer chains. It was found that cellulose was precipitated from the ionic liquid solution by the addition of water. When the water content of the ionic liquid was greater than about 1 weight percent (approximately 0.5 mole fraction H.sub.2 O), the solvent properties were significantly impaired and fibrous cellulose was found to be no longer soluble.

The production of ethanol from the fermentation of cellulosic materials such as grains is well known. The process is not efficient, but is capable of the production of very large quantities at a fairly reasonable cost. The process has the additional advantage of producing the product worldwide and shipping it to other markets as necessary. There are two significant features of the process: one is the need for heat. The fermentation process requires constant temperatures for the biological activity of enzymes and microorganisms to accomplish the conversion. The second feature is the production of carbon dioxide (CO.sub.2) from the fermentation itself, but also from the use of fossil fuels to heat the process. The capture of the CO.sub.2, also hereinafter referred to as “CO2”, is sporadic amongst producers, resulting in an overall significant contribution to the greenhouse gas pool from the industry.

The products of fermentation are ethanol and carbon dioxide, produced in 1:1 ratio as generally understood by those skilled in the art.

Biomass slurry is hydrolyzed in a fuel fired hydrolysis heater. When the biomass is fruit waste, the organic acid in the waste is used as the hydrolysis catalyst. When the biomass does not contain organic acid, carbon dioxide generated in a fermenter is fed to the hydrolysis heater as carbonic acid to provide the catalyst.

It is also widely known in the art that catalysts accelerate a wide range of chemical reactions. Such catalysts include dilute acid catalysts as selected from the group consisting of H.sub.2 SO.sub.4, HCl, HNO.sub.3, SO.sub.2 or any strong acid which effects pH values below about 3, and metal salt catalysts as selected from the group consisting of ferrous sulfate, ferric sulfate, ferric chloride, aluminum sulfate, aluminum chloride, and magnesium sulfate, Ni/Co, Rh/CeO.sub.2/M, where M represents SiO.sub.2, Al.sub.2O.sub.3 or ZrO.sub.2, Ni catalysts supported on zeolites (the use of zeolites as supports inhibited tar formation but promoted carbon deposition).

Pre-treatment—Since 1919, when Beckmann patented an alkaline pre-treatment based on impregnation with sodium hydroxide, which improved the digestibility of straw, many pre-treatments have been developed for lignocellulosic materials. Of the pre-treatments tested, hydrothermal processes appear to be among the most effective for improving the accessibility of these materials. An example of these hydrothermal processes is described in Shell International Research's Spanish patent ES87/6829, which uses steam at a temperature of 200-250 degree C. in a hermetically sealed reactor to treat previously ground biomass.

Discontinuous steam explosion treatment was patented in 1929 by Mason (U.S. Pat. No. 1,655,618) for the production of boards of timber, and it combines a thermal treatment with steam and the mechanical disorganization of lignocellulosic fibre. In this process, the wooden splinters are treated with steam at a pressure of 3.5 MPa or higher, in a vertical steel cylinder. Once the treatment is completed, the material is violently discharged from the base of the cylinder. This process combines the effects on the lignocellulosic material of high pressures and temperatures together with the final and sudden decompression. In the discontinuous steam explosion process developed by IOTECH Corporation, known as “flash hydrolysis”, the wood is ground to a small particle size and subject to temperatures and pressures close to 230 degree C. and 500 psi, and once these conditions are reached, it is suddenly discharged from the reactor.

Glucose can either serve as a feedstock for biochemical conversion (i.e., fermentation) to higher value products such as alcohol or organic acids, or it can be chemically converted (using catalytic processes) to products such as levulinic acid, sorbitol, and other polyols or glycols.

Researchers already have developed improved catalysts that enable cost-effective conversion of sugars that are not recovered during food processing into important chemicals called polyols. About four million tons of polyols are sold each year in the U.S., ultimately used in products like antifreeze, polyester fibers, cosmetics and plastics. Polyols can be produced from plant-based sugars much more energy-efficiently and cost effectively than from petroleum, which is how they are produced currently.

All forms of biomass have the same major components—cellulose, hemicellulose, and lignin. Cellulose is the largest fraction (40 to 50%), hemicellulose is next (20 to 30%) and lignin is usually 15 to 20% of biomass. The cellulose is composed of linear polymers of the six-carbon sugar glucose linked by 1,4 glycosidic bonds. Hemicellulose is a complex of primarily five carbon sugars, the majority of which are xylose and arabinose. Lignin is a complex polymeric heterogeneous material composed of variously substituted benzene rings.

Electricity is also a co-product of ethanol production generated at the rate of 2.28 kWh per gallon of ethanol or 68,692 MJ of electricity per hour. The energy value for ethanol and the co-product electricity is about 6×1011 MJ/year.

The stover conversion process generates both ethanol and electricity and requires a small amount of non-renewable energy for feedstock production, transport, conversion, distribution and delivery to the end user. Because of the electricity generation, the conversion process actually produces a negative flow of non-renewable energy usage of −0.109 MJ per mile driven for E100 as compared with 5.84 MJ non-renewable energy per mile for gasoline.

It is widely known in the art that acids aid the solubilization of cellulose including sulfuric acid.

It is also widely known in the art that enzymes must be stabilized, especially when utilized in supercritical fluids. Exemplary enzymes include immobilized CALB (Novozyme), as noted in the paper titled “Single-Enzyme Nanoparticles Armored by a Nanometer-Scale Organic/Inorganic Network” by Jungbae Kim et al. of Pacific Northwest National Laboratory, 902 Battelle Blvd., P.O. Box 999, Richland, Wash. 99352; where enzymes include cellulase (CELLUCLAST 1.5L, from the NOVO-NORDISK), and the .beta.-glucosidase enzyme is NOVOZYME 188 (NOVO-NORDISK). Immobilization can further increase the enzyme stability including the utilization of carriers as selected from the group consisting of silicas, zeolites, aluminas and kaolins.

It is also widely known in the art that utilization of high temperature resistant enzymes (e.g., heat-tolerant yeast Kluyveromyces marxianus CECT 10875) enhance the throughput and economics of fuel synthesis.

It is also widely known in the art that separation techniques include filtration recognized as microfiltration, ultrafiltration, and nanofiltration.

It is also widely known in the art that alternative fuels also include the production of methyltetrahydrofuran from the levulinic acid, catalytic cellulignin fuel (U.S. Pat. No. 6,855,180 for “Catalytic cellulignin fuel” to Pinatti, et al.) including furfural and levulinic acid from lignocellulose.

It is also known in the art that “Development of the Batch-Type and Flow-Type Supercritical Fluid Biomass Conversion Systems” by D. Kusdiana, E. Minami, K. Ehara, and S. Saka of Kyoto University International Symposium On Post-Petrofuels in the 21st Century Prospects in the Future of Biomass Energy, Sep. 3-4, 2002, Montreal, Quebec, Canada, pp. 276-279 has proven that the cellulose was hydrolyzed in the supercritical water to glucose in an extremely short time.

It is also known in the art that “Production of Liquid Alkanes by Aqueous-Phase Processing of Biomass-Derived Carbohydrates” by George W. Huber, Juben N. Chheda, Christopher J. Barrett, and James A. Dumesic, of Department of Chemical and Biological Engineering, University of Wisconsin at Madison, Madison, Wis. 53706, USA has proven that liquid alkanes are of the appropriate molecular weight to be used as transportation fuel components, and they contain 90% of the energy of the carbohydrate and H2 feeds. Thus, there has been much interest in processes that efficiently convert alkanes to alkenes.

It is also known in the art that photoirradiation has been used to activate several metal complexes.

Additional references include the following:

“High combustion activity of methane induced by reforming gas over Ni/Al2O3 catalysts” by Baitao Li, Ritsuko Watanabe, Kenji Maruyama, Mohammad Nurunnabi, Kimio Kunimori, and Keiichi Tomishige, published in Appl. Catal. A: General, 290, 36-45 (2005).

“Catalytic performance and properties of Ceria based catalysts for cyclic carbonate synthesis from glycol and carbon dioxide” by Keiichi Tomishige, Hiroaki Yasuda, Yuichi Yoshida, Mohammad Nurunnabi, Baitao Li, and Kimio Kunimori, published in Green Chem., 6, 206-214 (2004).

“Selective formation of ethylene carbonate from ethylene glycol and carbon dioxide over CeO2-ZrO2 solid solution catalysts” by Keiichi Tomishige, Hiroaki Yasuda, Mohammad Nurunnabi, Baitao Li, and Kimio Kunimori, published in Stud. Surf. Sci. Catal., 153, 165-168 (2004).

“Modeling of carbon-catalyzed gasification of organic feedstocks in supercritical water for energy conversion” at http://www.dieter-ulber.de/ Bachelor Thesis; Dartmouth College (1997).

Experiments of biomass gasification in supercritical water yielded a power cycle using wet biomass as a fuel, in which the process utilizes biomass (22 wt % in water) pumped up to 25 MPa in an extruder-feeder and heated up to 600° C. in a gasifier. There it is almost completely gasified into a medium heating value gas (15 MJ/kg) after about 30s residence time. The applied catalyst can be different types of charcoal or activated carbon. The efficiency of this biomass integrated-supercritical gasifier/combined cycle (BISG/CC) is calculated to be 42%.

“Direct synthesis of organic carbonates from the reaction of CO2 with methanol and ethanol over CeO2 catalysts” by Yuichi Yoshida, Yoko Arai, Shigeru Kado, Kimio Kunimori, and Keiichi Tomishige, published in Catal. Today, in press.

The use of carbon dioxide as a starting material for the synthesis of organic compounds has long been a goal for chemists. The hydrogenation of carbon dioxide to formic acid, methanol and other organic substances is particularly attractive, but has remained difficult. An efficient production of formic acid in a supercritical mixture of carbon dioxide and hydrogen containing a catalytic ruthenium phosphine complex is known in the art.

It is also known in the art, as in U.S. Pat. No. 6,875,456, hydrolyzed proteins by enzymatic hydrolysis from a variety of sources are used widely in the food industry, specifically as a means to provide flavorings. To liberate as many amino acids as possible, the enzymatic route employs complex mixtures of several endo- and exoproteases.

U.S. Pat. No. 6,509,180 and United States Patent Application No. 20030077771, for “Process for producing ethanol” to Verser, et al. produces ethanol with an acetate, acetic acid or mixtures thereof as an intermediate conversion product followed by the enzymatic hydrolysis of sugars and amino acids prior to fermentation. The '180 patent discloses the conversion of amino acids, again only as an intermediate conversion product into bacterial single cell protein.

It is further known that microwave-assisted enzymatic digestion is realized as depicted in a paper titled “Protein Preparation and Enzymatic Digestion in Proteomics” by Wei Sun et al. Another exemplary is depicted in another paper titled “Microwave-Assisted Hydrothermal Degradation of Silk Protein to Amino Acids” by Armando T. Quitain et al. at Research Institute for Solvothermal Technology, 2217-43 Hayashi, Takamatsu, Kagawa 761-0301, Japan, and Department of Ecological Engineering, Toyohashi University of Technology, Tempaku, Toyohashi 441-8580, Japan.

It is also known in the art in the paper titled “Effect of carbonate solvents on the conductivity and viscosity behaviour of ionic liquid”, by Boor Singh Lalia et al. Department of Applied Physics, G.N.D. University, Amritsar-143005, India, and Polymer Electrolyte Fuel Cell Research Department, Korea Institute of Energy Research, 305-343, Daejeon, Korea, that the effect of the addition of carbonate solvents, such as propylene carbonate (PC) and dimethyl carbonate (DMC) in an ionic liquid results in a decrease in the viscosity of the ionic liquid along with an increase in conductivity by more than one order of magnitude.

It is also known in the art that ionic liquids having the presence of water precipitates cellulose out of solution. The presence of water in the ionic liquid significantly decreases the solubility of cellulose, presumably through competitive hydrogen-bonding to the cellulose microfibrils which inhibits solubilization. Cellulose could be precipitated from the IL solution by the addition of water, or other precipitating solutions including ethanol and acetone. U.S. Pat. No. 5,846,393 for “Electrochemically-aided biodigestion of organic materials” to Clarke et al. also discloses the presence of water in the ionic liquid significantly decreases the solubility of cellulose. In this view, a paper titled “A new category of liquid salt-liquid ionic phosphates (LIPs)” by Robert Engel et al. notes that, unlike ionic liquids bearing tetrafluoroborate of tetrachloroaluminate anions, the LIPs are unreactive toward water. Further, the LIPs bearing simple phosphate anions are soluble in water, unlike their corresponding hexafluorophosphate salts.

It is also known in the art that a photocatalytic process particularly in combination with photosensitizing ions including photosensitizing ions selected from the group obtained from TiO.sub.2, ZnO, zinc, or WO.sub.3 leads to enhanced oxidative reactions. It is further known in the art that titanium oxide absorbs UV rays having a wavelength of about 400 nm or less, to thereby excite electrons thereof. When the resultant electrons and holes reach the surfaces of titanium oxide particles, the electrons and holes are combined with oxygen or water, thereby generating various radicals. The resultant radicals exert an oxidizing effect to thereby oxidize and decompose substances adsorbed on the surfaces of the particles. Furthermore, TiO2 prepared under calcination at 200° C. exhibited high photocatalytic activity for degradation of NOx under both ultraviolet (UV) and visible-light illumination. It is also known in the art that titania-supported copper plays a crucial role for promoting the reduction of CO2.

Another instance recognizing the potential of photocatalytic oxidation is in a paper titled “Aqueous Photocatalytic Oxidation of Lignin and Humic Acids with Supported TiO2” by Elina Portjanskaja et al. at Department of Chemical Engineering, Tallinn University of Technology, Ehitajate tee 5, Tallinn 19086, Estonia, and Department of Chemical Technology, Lappeenranta University of Technology, P.O. Box 20, 53851 Lappeenranta, Finland, where the addition of Fe2+ ions, up to 0.05 mM, to a lignin solution lead to a dramatic increase, about 25%, in photocatalytic efficiency.

It is also known as noted in biobleaching studies that the use of violuric acid is an effective mediator for laccase catalyzed deligification of kraft pulps. Violuric acid (2,4,5,6(1H,3H)-pyrimidine-tetrone 5-oxime, VOH) is often employed as an analytical reagent for chromatographic separation and for cation oxidation. It is also widely used in pulp bleaching techniques because the process is not very sensitive to temperature and pH variations. VOH can be also used as an efficient electron transfer mediator in oxidation processes allowing the increase of the global rate of electron transfer. Upon electrochemical oxidation the mediator violuric acid forms a radical with a lifetime on the order of several tens of minutes which oxidizes the lignin. The quality of the delignified pulp is remarkable due to the very high selectivity of the violuric acid radical in the oxidation of lignin over cellulose.

Another known method in the art is the methanol synthesis from carbon dioxide with a current efficiency of circa 90 by the electrolysis of carbon dioxide-saturated phosphate buffer solution in the presence of formate dehydrogenase and methanol dehydrogenase as electrocatalysts and pyrroloquinoline quinone as an electron relay.

An Internet link at http://pubs.acs.org/cen/science/83/8340scil.html?print notes Science & Technology, Oct. 3, 2005, Volume 83, Number 40, pp. 36-39, ACS Meeting News titled “Green Polymer Field Blossoming” discloses a clean solvent supercritical carbon dioxide and a natural catalyst, that is, an enzyme, makes unusual block copolymers. The composites consist of poly(lactic acid), derived from cornstarch, grafted onto cellulosic nanowhisker fillers produced by acid hydrolysis of cellulose. “Our approach uses reactive groups on the surface of the nanocellulosics to initiate the polymerization reaction of lactide,” Dorgan said. Dorgan's group has shown that ecobioanocomposites prepared from poly(lactic acid) and microcrystalline cellulose fillers have higher glass-transition temperatures.

A pretreatment process known in the art is depicted in the paper “Pretreatment for Cellulose Hydrolysis by Carbon Dioxide Explosion”, by Yizhou Zheng et al. at the Laboratory of Renewable Resources Engineering, 1295 Potter Engineering Center, Purdue University, West Lafayette, Ind. 47906, Accepted Sep. 21, 1998. Zheng et al. uses an explosive release of the carbon dioxide pressure to disrupt the cellulosic structure as a means of increasing the accessible surface area of the cellulosic substrate to enzymatic hydrolysis. Results indicated that supercritical carbon dioxide is effective for pretreatment of cellulose. An increase in pressure facilitates the faster penetration of carbon dioxide molecules into the crystalline structures, thus more glucose is produced from cellulosic materials after the explosion as compared to those without the pretreatment. This explosion pretreatment enhances the rate of cellulosic material hydrolysis as well as increases glucose yield by as much as 50%.

United States Patent Application No. 20060211096 for “Enzyme catalysis in the presence of ionic liquids” to Kragl, Udo et al. discloses a method for the conversion of substances in the presence of enzymes as a catalyst in a reaction medium comprising at least one ionic liquid, wherein the enzyme is selected from the group consisting of oxidoreductases, lipases, galactosidases, glycosidases, lyases and enzymes in EC class 6. It conclusively demonstrated that the presence of ionic liquids increases the yield above 55% when using lactose as an inexpensive donor.

All cited references, including patent and non-patent literature, are hereby incorporated by reference in their entireties.

The art lacks a high energy efficiency biomass fuel conversion solution with the additional inherent features of carbon dioxide sequestration by integrating a supercritical carbon dioxide hybrid absorption heat pump with integral power generating thermodynamic cycle.

### SUMMARY

A biomass to biofuel as a standalone plant and yet further integrated with a biomass to biodiesel plant process method having superior energy balance and higher value added co-products is provided. The process preferably uses an integrated carbon dioxide absorption heat pump and power generation cycle that utilizes a liquid, non-toxic absorbent such as ionic liquids, from which the carbon dioxide gas is absorbed, that further enhances the biomass hydrolysis process. The further incorporation of enhanced protein hydrolysis with the further advantage of debittering free amino acids and peptides provides for higher value added co-products rather than the traditional animal feed byproduct.

In one embodiment, the present invention is an ionic liquid hybrid solution utilized within thermal energy transformation devices. The devices use a solution comprised of ionic liquids that is an effective thermal transport media.

Additional combinations of refrigerants and absorbers are recognized in the art as having partial miscibility. A further aspect of the invention is the achievement of phase separation as a function of at least one function selected from the group consisting of temperature, pressure, and pH. The preferred solution further includes the utilization of small variations in pH to vary solubility of the refrigerant within the absorber. The more preferred solution varies temperature and pressure, in combination with pH control, using methods including electrodialysis. Additional methods to enable phase separation include the application of electrostatic fields, as electrostatic fields increase solubility of ionic fluids.

One aspect of the invention is to integrate an absorption heat pump with integral power extraction capabilities to a standard biomass pretreatment process.

The figures depicted within the specification provide exemplary configurations of the most important components of the biomass conversion system. A detailed description of the figures is provided in the following paragraphs.

### BRIEF DESCRIPTION OF DRAWINGS

FIG. 1 is a process flow chart view depicting an exemplary series of steps from biomass pretreatment process to energy generation.

FIG. 2 is a process flow chart view depicting another exemplary series of steps from biomass pretreatment to microchannel injection of supercritical water through carbon dioxide sequestration.

FIG. 3 is a process flow chart view depicting an exemplary series of steps integrating both thermal means and photocatalytic exposure leveraging the additional solar alternative energy.

FIG. 4 is a A process flow chart view depicting an exemplary integration of supercritical carbon dioxide absorption heat pump system with supercritical pretreatment of biomass.

FIG. 5 is a A process flow chart view depicting another exemplary direct integration of binary solution of supercritical carbon dioxide and ionic liquids biomass pretreatment.

FIG. 6 is a A process flow chart view depicting the direct integration of a biomass to biofuel with a biomass to biodiesel.

FIG. 7 is a A process flow chart view depicting the direct integration of biomass to biofuel pretreatment step with an absorption heat pump having power generation capabilities.

FIG. 8 is an overview of the inputs and outputs of the biomass to biofuel conversion process.

FIG. 9 is a process flow chart view depicting an alternative distillation process for dehydration of biofuel by operating the distillation process as a binary solution Organic Rankine power generation thermodynamic cycle.

### DETAILED DESCRIPTION OF THE PREFERRED EMBODIMENTS

The term “thermodynamic cycle” is defined as a process in which a working fluid undergoes a series of state changes and finally returns to its initial state.

The term “solar energy” is defined as energy derived from the sun, which most often refers to the direct conversion of radiated photons into electrons or phonons through a wide range of means. Solar energy is also indirectly converted into additional energy forms such as the heating of ground water (a.k.a. geothermal water).

The term “ionic liquids” “ILs” is defined as liquids that are highly solvating, non-coordinating medium in which a variety of organic and inorganic solutes are able to dissolve. They are effective solvents for a variety of compounds, and their lack of a measurable vapour pressure makes them a desirable substitute for Volatile Organic Compounds (VOCs). Ionic liquids are attractive solvents as they are non-volatile, non-flammable, have a high thermal stability, and are relatively inexpensive to manufacture. The key point about ionic liquids is that they are liquid salts, which means they consist of a salt that exists in the liquid phase and have to be manufactured; they are not simply salts dissolved in liquid. Usually one or both of the ions is particularly large and the cation has a low degree of symmetry. These factors result in ionic liquids having a reduced lattice energy and hence lower melting points.

The term “thermal tolerant” refers to the property of withstanding partial or complete inactivation by heat and can also be described as thermal resistance or thermal stability.

The term “pressure train” refers to independent pressure zones are alternatively produced by the utilization of flow control devices. One such device is a pressure relief valve. The utilization of a series of pressure relief valves, such that the cracking pressure is set incrementally to increase from the first pressure relief valve to the last with incremental increases for each pressure relief valve is an effective way to prevent backflow and to inherently control means to increase working fluid vapor state. The aggregate of the series of pressure relief valves within a heat exchanger is hereinafter referred to as a “pressure train” heat exchanger. Thus the pressure relief valve creates effectively independent zones within the pressure train. There are numerous methods known in the art to achieve precise and/or relative pressure control.

The term “heat pumps” refers to a device for delivering heat or cooling to a system, whereas a refrigerator is a device for removing heat from a system. Thus, a refrigerator may be considered a type of heat pump. Throughout the application, reference will be made to a thermal energy transformation device, hereinafter referred to as “TED” with the understanding that the designation of refrigerator, air conditioner, compressor, water heater, trigeneration, and cogeneration could be substituted without changing the operation of the device, specifically TEDs that utilize supercritical and transcritical fluids.

In absorption heat pumps, an absorbent, such as water, absorbs the refrigerant, typically ammonia, thus generating heat. When the combined solution is pressurized and heated further, the refrigerant is expelled. When the refrigerant is pre-cooled and expanded to a low pressure, it provides cooling. The low-pressure refrigerant is then combined with the low-pressure depleted solution to complete the cycle.

Ionic liquids and solids are recognized in the art of environmentally friendly solvents. Ionic liquids “IL” have very low if not negligible vapor pressure and are preferably selected from the group consisting of ionic liquids compatible with supercritical carbon dioxide “scCO2”. The inventive combination of scCO2 and ILs have excellent carbon dioxide solubility and simple phase separation due to their classification as partially miscible fluid combinations. Partially miscible fluids are both miscible and immiscible as a direct function of both pressure and temperature. A partially miscible fluid in its immiscible state can be simply decanted for phase separation, which is inherently a low energy separation method. The phase behavior of CO.sub.2 with ionic liquids and how the solubility of the gas in the liquid is influenced by the choice and structure of the cation and the anion.

The term “electride” is defined as being like alkalides except that the anion is presumed to be simply an electron which is localized to a region of the crystal between the complexed cations.

The term “supercritical” is defined as the point at which fluids have been exploited above their critical temperatures and pressures.

The term “heat pump” is defined as the transport of thermal energy extracted from a heat source to a heat sink by means including vapor compression, absorption, and adsorption.

The term “electron acceptor” is a compound that receives or accepts an electron during cellular respiration. The process starts with the transfer of an electron from an electron donor. During this process (electron transport chain), the electron acceptor is reduced and the electron donor is oxidized. Examples of acceptors include oxygen, nitrate, iron (III), manganese (IV), sulfate, carbon dioxide, or in some cases the chlorinated solvents such as tetrachloroethene (PCE), trichloroethene (TCE), dichloroethene (DCE), and vinyl chloride (VC).

The term “process intensification mixer” is defined as the utilization of micromixing, particularly with supercritical fluids, to achieve high mass transfer. Supercritical fluids include gases such as carbon dioxide, methane, methanol, ammonia, ethanol, butanol, and hydrogen. The devices include hydrodynamic cavitation devices, spinning disk, and spinning tube in tube.

The term “absorption” is widely accepted in the application of heat pumps for cooling. Absorption, in chemistry, is a physical or chemical phenomenon or a process in which atoms, molecules, or ions enter some bulk phase—gas, liquid or solid material. This is a different process from adsorption, since the molecules are taken up by the volume, not by surface. A more general term is sorption which covers adsorption, absorption, and ion exchange.

Pretreatment Efficiency Enhancements

The utilization of a biomass solution comprising the pretreatment step of solubilizing biomass solution in ionic liquids is an optimal means of producing alternative energy fuels. Ionic liquids have the distinct advantage of being both superior fluids for solubilizing cellulose, hemicellulose, and lignin from a variety of biomass sources. The preferred embodiment utilizes liquid ionic phosphates “LIPs”, polyammonium ionic liquid sulfonamides “PILS”, poly(ionic liquids), or combinations thereof, with the additional distinct advantage of reduced premature solids (i.e., cellulose, etc.) precipitation when the biomass solution has a significant (above 2%) moisture content. The fluid, which in this instance is an ionic liquid that solubilizes the biomass, is herein after referred to as the “solubilizing fluid”.

One specifically preferred embodiment combines the solubilizing fluid with at least one gas selected from the group consisting of carbon dioxide, ammonia, and methane. The benefits are particularly superior when the gas is pressurized to at least the supercritical pressure as a means of increasing mass transfer rates.

The integration of the solubilizing fluid, also interchangeably referred to as the working fluid, for pretreatment of a biomass and as an absorbent within an absorption heat pump/power generator has the further benefit of increasing the energy balance associated with the production of biofuels such as ethanol or butanol.

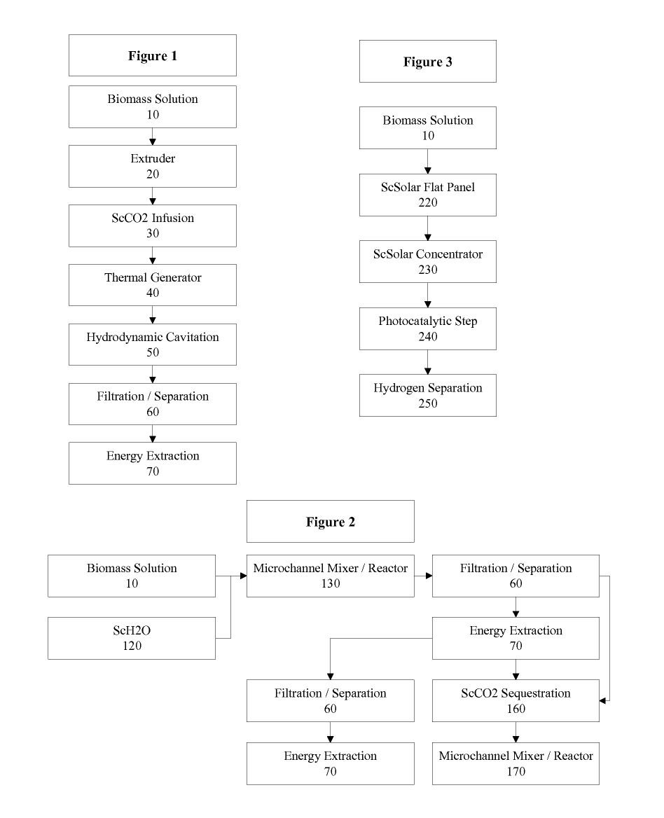

Referring to FIG. 1, the pretreatment process is depicted where the biomass solution 10 is preferably extruded 20 to a pressure equivalent to the pressure of the supercritical carbon dioxide “ScCO2” 30 that is absorbed into the solubilizing fluid phase of the biomass solution 10 as a supercritical liquid. The preferred source of the ScCO2 is desorbed from an integrated absorption heat pump. The utilization of an absorption heat pump greatly reduces (on the order of a 90% reduction) the electricity energy requirements as compared to traditional compression of CO2. The biomass solution infused with ScCO2 is further heated by a thermal generator 40, which can be anything from process waste heat of a power generating cycle, pyrolysis/gasification waste heat, to a traditional boiler, to the preferred hydrolysis temperature as known in the art and specific to the enzymatic and/or catalytic additives. The resulting biomass solution is further processed utilizing the preferred process intensification mixer, including the depicted hydrodynamic cavitation device 50 that has an additional benefit of creating very high instantaneous pressures during the collapse of bubbles thus creating cavitation. A wide range of equipment is known in the art for achieving hydrodynamic cavitation including an exemplary system as provided by VRTX Technologies LLC of San Antonio, Tex., USA. Hydrodynamic cavitation equipment reduces the biomass particle size resulting in increased surface area of the cellulose, hemicellulose, and lignin within the solution. The ultimate result being increased surface activity, whether the post treatment processes, as known in the art, includes the catalytic or enzymatic breakdown of the cellulose, hemicellulose, and lignins into fuel intermediaries. A filtration and separation process step utilizing the preferred micro- and/or nano-filtration membranes 60 are utilized to isolate soluble components from in-soluble solid components, and subsequently undergo the traditional explosion process to further break the hydrogen bonding present in the cellulosic structure. The preferred embodiment extracts the available enthalpy from biomass solution via an energy extraction device 70, with the particularly preferred devices selected from the group consisting of gerotors, pressure exchanger, turbines, quasiturbines, pistons, and ramjet as a means of increasing the energy efficiency of the fuel production process. The particularly preferred expansion devices are gerotors and ramjets, both having the advantage of high expansion efficiency and low damage susceptibility to precipitated cellulose and it's byproducts. Yet further means of increasing the overall system efficiency includes the selection of high efficiency components for the expansion of ScCO2 stage including the utilization of high efficiency gerotor, mechanical energy extraction device including gerotor, expansion turbine, expansion pump, Stirling cycle engine, Ericsson cycle engine, ramjet turbine, or combinations thereof. The particularly preferred energy extraction devices are integral supersonic devices selected from the group consisting of gerotor, compressor and turbine including compressors and turbines operating on either the ramjet or pulsejet principle.

Also referring to FIG. 1, numerous means are known in the art to increase the pressure of the biomass solution, though the preferred is an extruder 20 having the benefits of both reaching the desired pressure of the non-compressible fluid with high energy efficiency (compressing a non-compressible fluid requires significantly lower energy than a compressible fluid, i.e. known in the art advantages of any absorption heat pump vs. a vapor compression heat pump). The further infusion 30 of the supercritical carbon dioxide at the absorption pressure (which is post the expansion device/evaporator) into the biomass solution enables the ScCO2 to be absorbed into the ionic liquid. A subsequent mechanical means is used to further raise the pressure of the biomass, ionic liquid, and carbon dioxide slurry to the generator/desorber pressure (i.e., high-pressure side of the thermodynamic cycle). The mechanical means include, though are not limited to, positive displacement pump, extruder, thermal hydraulic compressor/pump, or combinations thereof. The utilization of the ionic liquid has the principal advantage of concurrently enabling the rapid degradation of the cellulose, hemicellulose, and lignin products to byproducts capable, as known in the art, of being catalytically or enzymatically converted to a wide range of combustible fuels, and the integral functionality of the high ScCO2 absorption enabling high efficiency power conversion. The absorption power generation cycle can be either the primary energy generation cycle, a bottom cycle to other power generation thermodynamic cycles principally increasing the energy efficiency of the primary power generation cycle through energy recovery, a multi-effect absorption heat pump cycle, or incorporated into virtually any thermodynamic cycle driven by a thermal source.

The desorption “thermal generator” 40 stage is another net consumer of energy. The energy consumption (i.e., desorption temperature) can be decreased by means including the spinning disk reactor as a means of increasing the rate of heat transfer into/out of the biomass solution thus accelerating the absorption/desorption rate (thermally limited rates). Further means of increasing the high-side pressure include the utilization of a thermal-hydraulic compressor including a pressure train heat exchanger, a series of independent pressure stages having staggered or pulsed flow, hydraulic pump having an integral thermal sink, or combinations thereof. The biomass solution is desorbed at higher efficiencies by utilizing the combination of at least one thermal method and at least one non-thermal method including non-thermal methods selected from the group consisting of magnetic refrigeration, vapor compression heat pump condenser, solar activated direct spectrum light absorption, electrodialysis, electrostatic fields, membrane separation, electrodesorption, pervaporation, gas centrifuge, vortex tube CO2-liquid absorber, decanting, or combinations thereof. The utilization of fluids in combination with the biomass solution having regions ranging from miscibility, partial miscibility, or immiscibility enable high efficiency phase separation to be achieved by the varying operating parameters including at least one function selected from the group consisting of temperature, pressure, and pH.

Pretreatment of cellulose as a means to yield glucose is well established in the art, predominantly utilizing the energy intensive step of steam explosion. The inventive utilization of processing the biomass into an ionic liquid enables a significant reduction in thermal energy and a lowering of the reaction temperature requirements. The subsequent raising of the pressure of the biomass and ionic liquid slurry is achieved through mechanical means. The resulting intermediate pressure biomass solution is preferably where the intermediate pressure is equivalent to the integral absorption cycle low-pressure stage (i.e., absorber pressure).

Another particularly preferred embodiment is further comprised of microwave irradiation to increase the hydrolysis rate by a minimum of 10%. A specifically preferred embodiment immobilizes the enzymes within the working fluid by taking advantage of the ionic liquid's superior and specific absorption of microwave irradiation such that the enzymatic hydrolysis is enhanced by achieving a localized active catalytic center resulting in a reduction in the hydrolysis temperature of at least 5 degrees Fahrenheit lower than the pretreatment process void of microwave irradiation. The net result is reduced damage to proteins by thermal denaturing.

When the pretreatment biomass conversion process is either enzymatic or requires a temperature increase beyond the temperature at which proteins denature or enzymes lose their activity, the further inclusion of trehalose enhances the effective enzyme lifetime and limits protein denaturing. Numerous distinct advantages are present when the conversion of biomass to fuel is preferably achieved using catalytic reactions, with the preferred catalysts selected from the group consisting of sub-micron catalysts, sub-micron electrocatalysts, or sub-micron photocatalysts where sub-micron and nanoscale are used interchangeably. It is known in the catalytic reaction art that high surface area nanoscale (i.e., sub-micron crystallite particle size) and either photon or electron activation accelerates the reaction chemistry leading to both higher energy efficiency and decreased capital amortization costs. The distinct combined advantages realized by the preferred solubilizing fluid, the integration of the absorption heat pump for low energy requirement to achieve supercritical pressures, the integration of energy extraction to transform enthalpy into a useful co-product of electricity, and high mass transfer of supercritical reactions yield superior conversion of biomass to a wide range of resulting products from nanocomposite polymers to biofuels.

Referring to FIG. 3 is another embodiment of the pretreatment process where the particularly preferred reaction includes a photocatalytic process step 240 to further modify the biomass byproducts through the step of splitting hydrogen from the biomass solution, with a subsequent step of separating the hydrogen gas 250 by means known in the art. A specifically preferred method of processing the supercritical biomass solution 10 is further comprised of process steps to heat the biomass solution by solar means. A superior method is to utilize both supercritical solar flat panels 220 or supercritical solar concentrator receivers 230, whereby the optimal performance is achieved by configuring the solar devices in a sequential flow first into the supercritical solar flat panels and then into supercritical solar concentrator receivers as a means of minimizing capital cost and operating thermal losses.

The principal motivation for biomass to biofuel conversions, in other words the production of alternative energy fuels, is the reduction of global warming gases. The creation of global warming gases is largely influenced and is a function of the energy balance associated with the production process. Thus, the further inclusion and direct integration of an absorption heat pump having at least one working fluid component in fluid communication with the pretreatment process enables a reduction in energy consumption throughout the biomass conversion process and most notably in the energy intensive pretreatment process. The further benefit of the integration of the absorption heat pump is the low energy production of supercritical gases, particularly CO2 as absorbed into a wide range of refrigerant absorbents including glycolic acid, alcohols, amyl acetate, isobutyl acetate, ILs, LIPs, and PILs. The reduced energy requirement is attributed to the reduced electrical requirement of “compressing” a liquid as compared to compressing a gas.

The combined low energy process results in an increased biomass surface area to accelerate the hydrolyzing, oxidizing, and/or reducing reactions of the biomass solution. The supercritical gas (i.e., absorption heat pump refrigerant) is optionally further integrated into the biomass conversion process as a means of reducing the moisture content that is naturally present in biomass to limit the premature precipitation of cellulose and hemicellulose from the pretreatment working fluid. The supercritical gas, most notably CO2, is then subsequently dehydrated into glycerine or glycerol (working fluid component A1, which is a byproduct of the biomass to biodiesel conversion process). This dehydration process is significantly less energy intensive than traditional drying means of biomass, with the preferred moisture/water content of less than 2% on a weight basis of the working fluid. The moisture saturated glycerine/glycerol is regenerated by at least in part utilizing the recovered waste heat from at least one working fluid A2 (which in this example is supercritical CO2) component in fluid communication with both the biomass to biofuel conversion process and biomass to biodiesel conversion processes.

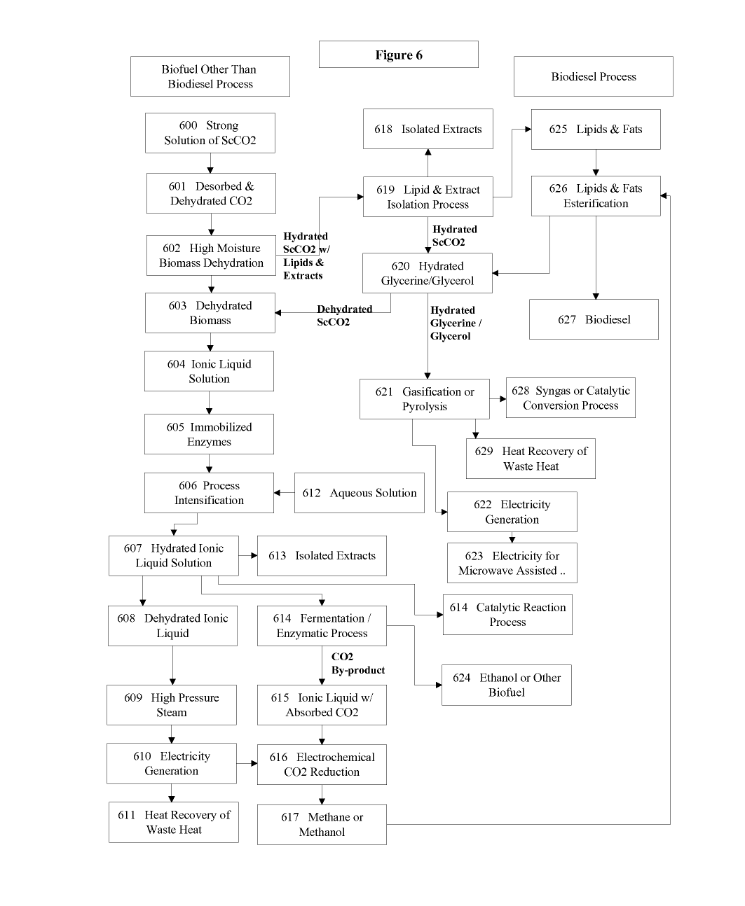

Referring to FIG. 6 is an embodiment having true integration of a biomass to biofuel with a biomass to biodiesel conversion process. The biofuel process is characterized as being comprised of a supercritical CO2 strong solution 600 that is desorbed, preferentially from an absorption heat pump, and dehydrated 601 by the infusion of the hydrated ScCO2 into the byproduct glycerine/glycerol from the biodiesel process. This transforms the high moisture biomass 602 into a reduced moisture content biomass solution 603 having increased compatibility with the aforementioned solubilizing fluid. The ScCO2 further contains lipids and extracts from biomass that are processed via an isolation/extraction process 619 as known in the art yielding high value add co-products 618 and lipids 625 utilized within the biodiesel process to be esterified 626 into biodiesel 627. The hydrated glycerine/glycerol 620 can either be regenerated for reuse or is pyrolized/gasified 621 into either syngas or further catalytically processed 628 to additional value add co-products. This pyrolysis/gasification stage 621 creates significant waste heat that can be recovered for multiple purposes via heat recovery system 629 including input thermal energy to the biomass to biofuel conversion process or the production of electricity 622, preferably via the aforementioned absorption heat pump/power generating cycle as thermal input into the generator/desorber. The electricity produced 622 is optimally utilized for various electrochemical processes and/or creating microwave irradiation 623 as a means of increasing the rate of hydrolysis within the aforementioned hydrolysis process. Furthering the biofuel conversion process is the isolation of the solubilizing fluid, preferentially comprised of ionic liquid solution 604 having immobilized enzymes 605. The pretreatment process of hydrolysis continues until such time as an aqueous solution 612, preferentially further comprised of electron transfer mediators, etc., is mixed via a process intensification mixer 606 creating a hydrated ionic liquid solution 607 yielding isolated extracts 613. The water component of the hydrated solubilizing fluid creating “desorbed” high pressure steam 609 which in turn produces additional electricity 610 again producing waste heat 611 that is utilized within the aforementioned AFEX and/or absorption heat pump cycles. Additionally, as a result of the desorbtion of water from the solubilizing fluid is the desorbtion of ScCO2 that is transformed into co-products via either a catalytic reaction process 614 or is a feedstock to a subsequent fermentation/enzymatic process 614. The fermentation/enzymatic process 614 yields additional CO2 which is absorbed into the solubilizing fluid (in the weak solution state) 615. The now strong solution is electrochemically reduced 616 creating methane/methanol 617, wherein the electrochemical process is driven off the generated electricity 610.

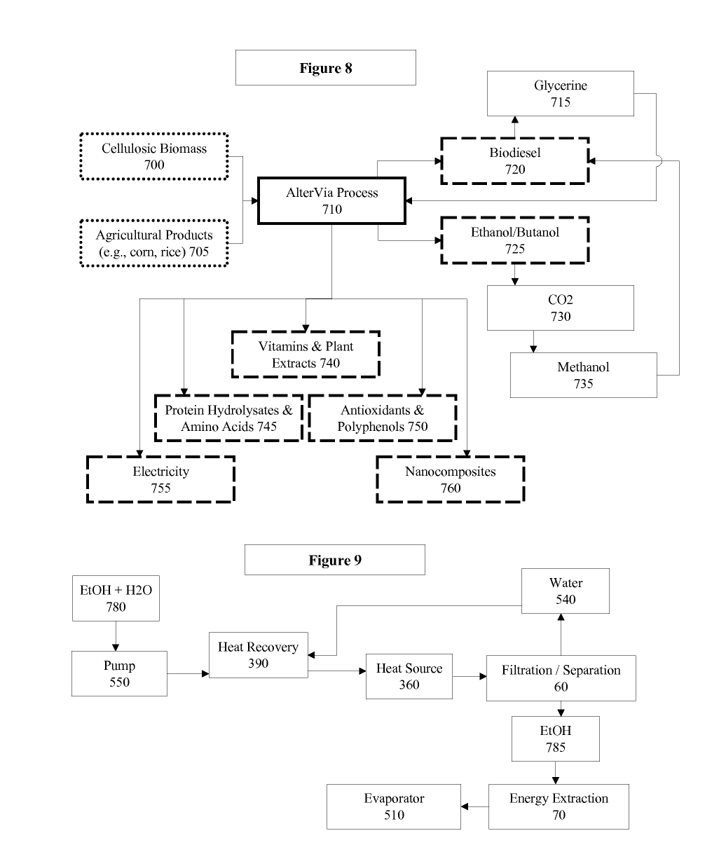

Referring to FIG. 8 is the overall raw material inputs and resulting products and co-products by implementing the aforementioned integrated biofuel and biodiesel processes, which is referred to as the AlterVia process 710. A cellulosic biomass 700 or agricultural products 705 (most notably products with a significant protein content) are raw material inputs. The first direct output is biodiesel 720 with its byproduct of glycerine 715 that is utilized as an input on the biofuel process side as characterized earlier. The second direct output is a biofuel including ethanol or butanol 725 with its byproduct of CO2 730 that is further processed by electrochemical reduction into methanol 735 and becomes an input on the biodiesel process side as characterized earlier. Additional co-products include isolated extracts such as vitamins and plant extracts 740, protein hydrolysates and amino acids/peptides 745, antioxidants and polyphenols 750. The further byproducts of waste heat are transformed into electricity 755, preferably by the aforementioned absorption heat pump/power generator. And lastly, the cellulosic fibers processed by the earlier characterized pretreatment process and microchannel precipitation process results in cellulosic nanowhiskers that are further processed into nanocomposites 760.

Secondary Efficiency Gains—The subsequent infusion of carbon dioxide, especially supercritical carbon dioxide “ScCO2”, has the secondary benefit of enhancing the biomass hydrolysis process. The preferred biomass solution is pressurized to a pressure in excess of 600 psia. A particularly preferred biomass solution is pressurized in excess of the supercritical pressure of carbon dioxide of 1073 psia, such that the biomass solution is within the supercritical region. The benefits of operating within the supercritical range has many significant benefits as known in the art including reduced surface tension, thus enabling the further utilization of microchannel heat exchangers, microchannel reactors, and the high reactivity of supercritical fluids for both catalytic and enzymatic chemical transformations. A source of carbon dioxide, as a further means of reducing the carbon dioxide greenhouse effect, is the integration of the fermentation byproduct of carbon dioxide being absorbed by the ionic liquid. It is further anticipated to incorporate the high efficiency biomass conversion system into alternative biomass to fuel conversion methods; additional power generation, industrial processes, waste treatment plants, or additional facilities that produce either waste heat or carbon dioxide. Therefore the inefficiencies and byproducts of one cycle are thus leveraged into the adjoining cycle providing real economic and greenhouse benefits beyond the operation of either single cycle system.

A particularly preferred absorption heat pump is further comprised of a power generation cycle to produce electricity utilized for at least one function selected from the group consisting of microwave irradiation, electrochemical reduction, and electrolysis. The direct integration of the power generating cycle has the means to reduce the cost of electricity required to implement a series of critical process steps to enhance the biomass conversion process while also producing waste heat recovered from the bottom cycle, which becomes in fluid communication with the biomass pretreatment process.

Another benefit of integrating the rapid expansion pretreatment process having an expanded gas and a power generating thermodynamic cycle, wherein the expanded gas from the pretreatment process is in fluid communication with the condenser of the power generating thermodynamic cycle, is increasing the thermodynamic cycle efficiency of both the biomass pretreatment process and the power generating thermodynamic cycle. The expanded gas increases the temperature differential within the thermodynamic cycle, thus enabling a higher Carnot efficiency. The energy recovery process is further comprised of a waste heat recovery device to recover thermal energy from the condenser of the power generating thermodynamic cycle whereby thermal energy is further increased by the heat of absorption by the subsequent mixing of the expanded gas into at least one absorbent prior to recombining with biomass solution. The temperature lift achieved by the heat of absorption increases the “quality” of the working fluid such that the thermal energy is utilize, at least in part, as a preheating stage. The waste heat is utilized for at least one function selected from the group consisting of preheating the inputs of the rapid expansion pretreatment process, thermal hydraulic pump, and inputs of an absorption heat pump as a means of increasing fluid pressure.

Yet another embodiment is the utilization of a waste heat recovery device to recover thermal energy from the condenser of the power generating thermodynamic cycle in fluid communication with the biomass solution and wherein the thermal energy is utilized as at least a partial thermal energy source within an endothermic reaction. The utilization of low “quality” waste heat as a first stage preheat in an endothermic reaction (such as glucose pyrolysis) increases the combined cycle energy efficiency.

Another embodiment leverages the advantages of binary fluids concurrently within the biomass pretreatment process and power generating cycle. Binary fluids achieve superior energy efficiency within Organic Rankine cycles, while the same binary fluids increase the hydrolysis rate within the biomass pretreatment process. Thus the power generating thermodynamic cycle is comprised of a working fluid having at least a first working fluid W1 and a second working fluid W2. Exemplary combinations for binary fluids are selected from the group consisting of carbon dioxide, ammonia, methanol, ethanol, butanol, and water. Particularly preferred combinations are CO2 and NH4, CO2 and methanol, CO2 and ethanol, or CO2 and butanol. The preferred thermodynamic cycles in which the benefits will be realized include cycles selected from the group consisting of Goswami, Uehara, Kalina, Rankine, Carnot, Joule-Brayton, Ericsson, and Stirling cycles. The preferred cycles are combination cycles in which the biomass conversion system leverages both waste heat and synergistic utilization of ionic liquids from any single thermodynamic cycle into a hybrid high efficiency thermodynamic cycle. A particularly preferred operating mode for the power generating thermodynamic cycle is selected from the group consisting of binary Organic Rankine, Goswami, Kalina, and Carnot cycles. The result is maximum power generation, overall energy efficiency, and reduced CO2 emissions.

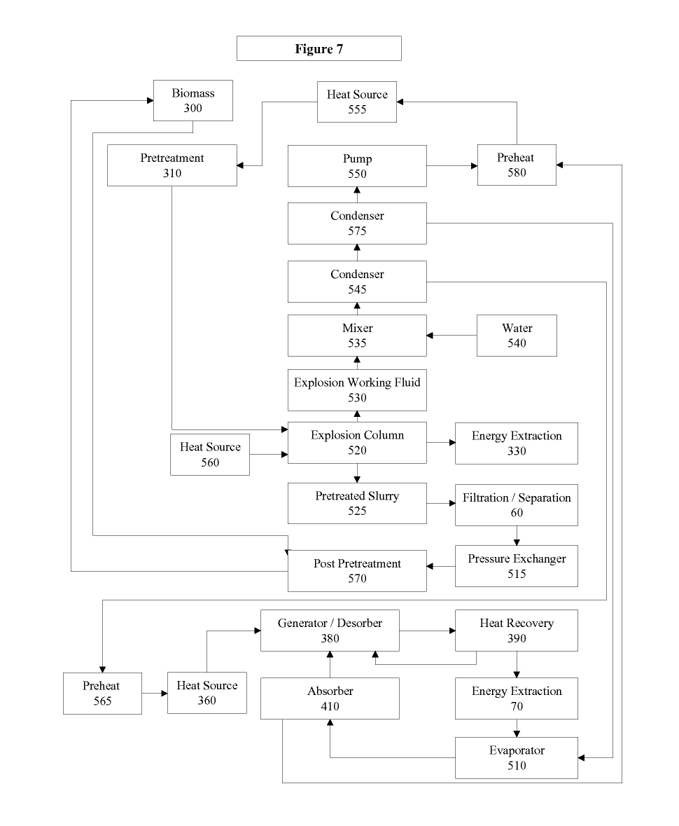

Referring to FIG. 7, is another embodiment that depicts a significant reduction of energy requirements beyond the improved ammonia fiber explosion “AFEX” process. The improved AFEX process is characterized as follows: Biomass 300 enters the pretreatment process 310 after being infused with thermal energy from an external source both within the pretreatment stage 310 from heat source 555 and with the explosion column 520 from heat source 560. The biomass solution is separated into two streams of pretreated slurry 525 and explosion working fluid 530 (which is predominantly ammonia in the AFEX process). The explosion working fluid 530 is subsequent infused with water 540 and mixed 535, which triggers the creation of thermal energy from the heat of absorption. The AFEX process then sequentially goes through two condensers, with the first being a traditional cooling tower 545 and the second requiring active cooling condenser 575 (thus the evaporator of a chiller) so that the combined water and ammonia solutions returns to a liquid requiring less energy to pump 550 the liquid, rather than compress a gas. A preferred embodiment integrates an absorption heat pump/power generating cycle as characterized by generator/desorber stage 380 with subsequent heat recovery 390 that preheats the strong solution entering the generator, the subsequent expansion of the desorbed working fluid (preferably ScCO2 or supercritical ammonia) with integral energy extraction device 70 producing a low temperature expanded gas (i.e., producing cooling) in the evaporator 510. The particularly preferred embodiment integrates the AFEX and absorption heat pump having fluid communications at various points. The first improvement is such that waste heat is recovered from condenser 545 that is utilized at least as part of the preheat 565 utilized prior to the heat source 360 (if even necessary dependent on desorption temperature). The second improvement is such that the cooling produced by the absorption heat pump made available to AFEX via evaporator 510 displaces the otherwise requirement for mechanical active cooling equipment. The third improvement is the direct integration of an energy extraction device 330 capable of handling the high solids content while concurrently extracting energy during the rapid expansion stage. Another improvement is the utilization of a pressure exchanger 515 following the filtration/separation membrane 60 wherein the solids are further process by a post pretreatment process 570, such that pressure is recovered from the pretreatment process with the non-pretreated biomass 300 to complete the cycle.

Another significant use of electricity is the conversion of the biomass conversion process byproduct of CO2 from the fermentation (or even gasification/pyrolysis) steps. The CO2 byproduct with H₂O is electrochemically reduced into methanol, which is then subsequently utilized as an input in the preferentially integrated biodiesel esterification process. This reduction reaction is best achieved within ILs, PILs, and/or LIPs due to the significant electrical conductivity in combination with the high CO2 absorption. A further means of increasing the electrical conductivity and decreasing the working fluid viscosity is by adding carbonate solvents including propylene carbonate “PC” and dimethyl carbonate “DMC”. Alternatively, the absorbed CO2 can be further processed by means including reactions of polymerizing carbon dioxide or carbonate synthesis as an energy effective means of sequestering CO2.

The high electrical (and thermal conductivity which enhances heat transfer) conductivity makes the biomass solution superior for a wide range of chemical reactions particularly those enhanced by electrochemical, electrolysis, electrocatalytic, or photocatalytic process steps. The further inclusion of nanoscale conductors and semi-conductors as a means of increasing quantum mean free path, quantum dots, copper, Fe2+ ions, iron-sulfur cluster, or electrides increase the reaction rates. The biodigestion of organic materials can be further enhanced by electrochemical process steps.

The presence of electron donors within the biomass conversion process, particularly when electrochemical processing is utilized, reduces the oxidation rate of hydrolysis. Thus removing sources of electron donors prior to hydrolysis including lignin, antioxidants, polyphenols, and aromatic compounds increases the photocatalytic or enzymatic oxidation rate by a minimum of 10% throughout the conversion/degradation occurring during fermentation of biomass solution to biofuels including ethanol and butanol. Electron donor sources include tocopherols, antioxidants, aromatics, etc.

Additional means of enhancing the conversion process is achieved by inclusion of at least one working fluid additive selected from the group consisting of electron transfer mediator including iron salts, derivatives of iron salts, potassium salts, lactic acid salts, derivatives of potassium salts, derivatives of lactic acid salts, phytic acid, gallic acid, potassium ferricyanide, polyoxometalates, violuric acid, polycationic protein, thialoto-bridged complexes, thiolated complexes, metalloproteins, protein complexes having an iron-sulfur cluster, trehalose complexes, iron-sulfur cluster, sodium-ammonia, sulfur-ammonia, a chitosan complex including chitosan lactate, chitosan alpha lipoic acid, and thiolated chitosan, nanoscale catalyst, electrocatalyst, photocatalyst, electron donor, electron acceptor, ultraviolet absorber, infrared absorber, quantum dot, nanoscale powder, enhancing electron transfer including iron salts, derivatives of iron salts, potassium salts, lactic acid salts, derivatives of potassium salts, derivatives of lactic acid salts, phytic acid, gallic acid and combinations thereof.

The direct integration and interdependency between the absorption heat pump, power generating capabilities, heat transfer, and numerous resulting co-products requires control systems well beyond the automation process requirements of traditional biomass conversion processes. Implementing a control system having non-linear algorithms capable of determining the maximum operating revenue in real-time by monitoring at least one parameter selected from the group consisting of cost and price of electricity, cooling cost and price per btu, heating cost and price per btu, carbon dioxide emission credits, cost and price of methanol per btu, cost and price of resulting biofuels per btu, conversion factor of electricity for electrochemical reactions, and operating parameters of a reverse fuel cell for electrochemical reduction of at least one component of the biomass solution results in optimization of revenue generation minus operating expenses.

Yet another embodiment is the integration of an energy extraction device within the rapid expansion step occurring within explosion steps (ammonia fiber explosion, steam explosion, supercritical explosion). The result is the concurrent production of electricity and cooling which terminates the hydrolysis process. The preferred energy extraction device includes a gerotor, pressure exchanger, and quasiturbine. These devices have the distinct advantage of enabling the pressure expansion with minimal impact of the biomass solids. Additional means for reducing the particle size of precipitated cellulose, which also increases surface area, utilizes a microchannel device having channels less than 10 microns prior to the expansion stage. A preferred embodiment utilizes a rapid expansion step that occurs in a series of independent pressure drop stages comprised of at least a first pressure drop stage and a second pressure drop stage. A particularly preferred pre-expansion pressure is a pressure greater than the fluid's supercritical pressure. The first pressure drop stage has a pressure below at least one working fluid's supercritical pressure. The utilization of the at least two pressure drop stages enables the maximum energy generating capability (i.e., transform thermal energy enthalpy of biomass solution into electricity) while minimizing viscosity issues associated with complete pressure letdown of the post-pretreatment biomass solids. The second pressure drop stage occurs within a pressure exchanger with high pressure fluid being the biomass solution from the exit of the pretreatment process and low pressure fluid being the biomass solution prior to the pretreatment process, as a means of further increasing the energy balance of the biomass conversion process.

Referring to FIG. 9 is another embodiment that further improves the energy balance by transforming the traditional distillation process for the biofuel process (specifically ethanol) into an energy producing step having higher efficiencies than traditional single cycle electricity power plants. The non-dehydrated/non-anhydrous ethanol “EtOH” 780 is preferably pumped 550 to a pressure above the supercritical pressure of at least one of the components within the EtOH and H₂O solution. The solution 780 is preheated 390 from thermal energy recovered from the water vapor/liquid 540 isolated by means known in the art including nanofiltration 60 and subsequently heated by a second stage heat source 360. The combined fluid is now operating in the mode of a binary solution Organic Rankine cycle having an anticipated operating efficiency near 30% while concurrently yielding pure EtOH 785 high pressure supercritical fluid that is expanded through an energy device 70 and further evaporated 510, preferably by the evaporator of the aforementioned absorption heat pump.

Yet another embodiment is the infusion of at least one working fluid additive selected from the group consisting of monomers, polymers solubilized in the at least one working fluid, microspheres, and nanoscale powders having particle size less than 100 nanometers. The particularly preferred additives are further comprised of immobilized enzymes, immobilized catalysts, or combinations thereof. Superior additive distribution is achieved by mixing the biomass solution with additives by at least one process intensification mixer including hydrodynamic cavitation devices, spinning disk, and spinning tube in tube. The utilization of microspheres serves multiple purposes including immobilizing enzymes for easy reuse and recovery of enzymes, reducing agglomeration of biomass solids post-pretreatment, reducing nanocomposite density while increasing polymeric strength. The resulting biomass solids, most notably cellulose, are further processed into polymers, copolymers, or block copolymers.

Carbon Dioxide Sequestration—Another feature of the inventive biomass conversion system is the subsequent processing the desorbed carbon dioxide post expansion, as a means of sequestering the carbon dioxide byproduct including means of polymerizing carbon dioxide or carbonate synthesis. Utilizing the desorbed carbon dioxide, which remains a high-pressure heat transfer fluid, continues to have relatively low surface tension enabling chemical reactions to take place within a microreactor.

Supercritical Working Fluids—Yet another feature of the biomass conversion system is the utilization of binary and even ternary solutions, recognized in the art, having the ability to enter into regions whereby the solution components vary from miscible, partially miscible, to immiscible range. These variations enable low energy methods of separating the solution within the immiscible range by simply decanting, centrifuging, or otherwise isolating the immiscible fluid components. Methods of transitioning between the miscible to immiscible range are accomplished by varying at least one parameter selected from the group consisting of pressure and temperature. Thus the ScCO2, which is the preferred heat transfer fluid is isolated from the biomass solution, for utilization within the thermodynamic cycle as a means of producing heating, cooling, power, or combinations thereof with the inventive integration of the biomass conversion process with a ScCO2 absorption heat pump system. A preferred working fluid for the absorption thermodynamic cycle is an ionic liquid, though an integrated bottom cycling absorption/desorption cycle is efficiently performed utilizing binary fluids comprised of at least materials selected from the group consisting of organic liquids, alcohols, ammonia, water, carbon dioxide, lithium chloride/bromide or combinations thereof.

A particularly preferred binary fluids are supercritical fluids. The maximum pressure of the supercritical biomass solution is significantly in excess of 600 psia. The high side pressure is a minimum of 1400 psia when the binary composition is isobutyl acetate or amyl acetate. The specifically preferred pressure is up to 5,000 psia for ionic liquids that have thermal stability up to 450 degrees Celsius.

Referring to FIG. 2, the further inclusion of water 120, especially when operating at a maximum pressure in excess of the supercritical pressure required for water, enables the additional benefit of enhanced conversion rate into fuels. Thus a specifically preferred biomass solution 10 is comprised of at least fluids selected from the group consisting of ionic liquids, carbon dioxide, and water. A preferred implementation mode is the mixing of the supercritical carbon dioxide, ionic liquid, and biomass with the supercritical water within a microchannel heat exchanger 130. The utilization of the microchannel heat exchanger generally minimizes the particle size of the precipitated cellulose to less than about 10 microns. Reaction products are then optionally separated immediately following microchannel reactor by separation methods known in the art 60. Another subsequent separation process occurs post the first stage of energy extraction 70, which then further goes through an energy extraction 70 that in this instance is ideally a pressure exchanger. The desorbed ScCO2 is sequestered 160 and further processed in a preferred embodiment into a high value added co-product by being chemically transformed within a high throughput microchannel mixer/reactor (a.k.a. process intensification mixer/reactor) 170. Alternatively or immediately prior to the microchannel heat exchanger is the mixing of the supercritical carbon dioxide, ionic liquid, and biomass with the supercritical water by hydrodynamic cavitation, which also has the benefit of intimate mixing virtually instantaneously.

Absorption Cycle Integration—Solubilizing the biomass at a temperature not exceeding 60 degrees Celsius enables the absorber to be “cooled” by the ambient temperature biomass as a means of increasing the efficiency from the low-temperature and low-pressure side of the absorption system. Creating said multiple stage effect absorption systems, as known in the art, further enhances both the absorption heat pump efficiency and the biomass to fuel conversion process.

The further integration of the absorption cycle and the biomass solution pretreatment process enables the expansion of the biomass solution to not only achieve rapid cooling for the subsequent quenching of the hydrolysis reaction, but also the concurrent extraction of energy (which can be either mechanical or electrical through methods known in the art of power generation). The solution is rapidly quenched by at least one process step selected from the group consisting of the sequential processing of hydrodynamic cavitation and expansion of the supercritical biomass solution, sequential expansion of the supercritical biomass solution to below water's supercritical pressure followed by the step of expansion of the supercritical biomass solution to below carbon dioxide's supercritical pressure. An optional step of performing carbon dioxide sequestration can be achieved at various points throughout the biomass conversion system (one such sequestration point is following the expansion of the biomass solution to below the point at which a significant water vapor component exists, which is largely a function of the post-ScCO2 step as known in the art ranging from chemical reactions producing carbonate products to polymerization. The introduction of the intermediary expansion stage enables the water to be isolated from the biomass solution as a further means of controlling the conversion rate of the biomass to fuel.

Separating components within the biomass solution is achieved by means including at least one method selected from the group consisting of nanofiltration, decanting of immiscible solution components, or combinations thereof. Each expansion stage has the further inclusion of energy extraction devices to produce mechanical or electrical energy and/or preceded respectively by filtration means as known in the art.

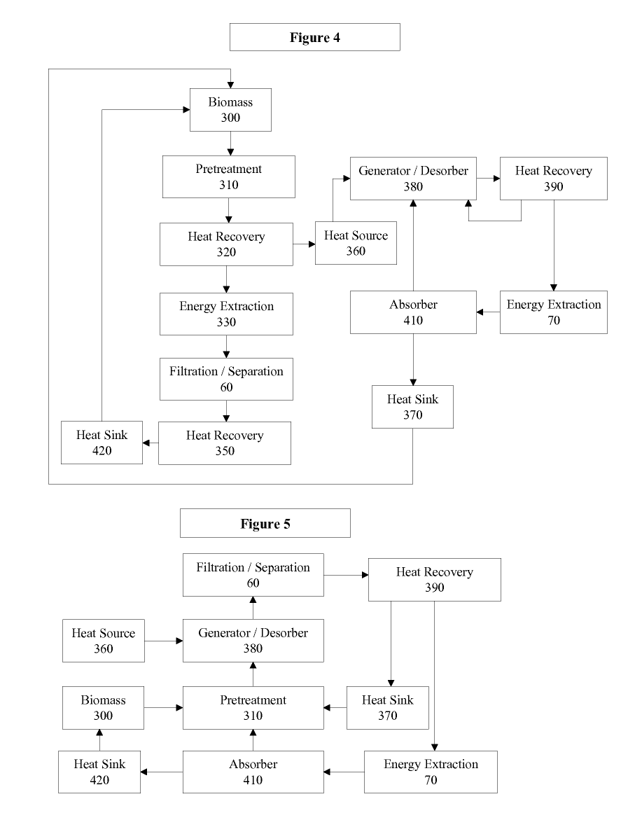

One exemplary layout is shown in FIG. 4 that discloses the multiple areas where heat transfer fluids through heat exchangers are in fluid communication between a biomass fuel conversion pretreatment process and an absorption heat pump system. The series of steps having heat transfer include: a) biomass is combined with ionic liquid into a biomass solution 300; b) Pretreatment step including raising the temperature and pressure of the solution by thermal means 310; c) Heat recovery 320 from the post pretreatment solution utilized as at least the first stage of providing thermal energy (heat source 360) for the absorption heat pump generator 380; d) Expansion through energy extraction device 330 of the pretreatment solution followed by filtration/separation of the byproducts 60, which can alternatively be prior to the expansion step; and e) Heat recovery 350 from the end product of the pretreatment biomass solution through a heat exchanger as a heat sink 420 which is utilized to preheat the biomass as a means of reducing thermal energy requirements, and additionally preheating biomass from thermal energy recovered from the absorption heat pump absorber 410 which also includes heat of absorption in addition to thermal energy transferred during the absorption cooling evaporator/energy expansion device 70 transferred through a heat exchanger heat sink 370 known in the art. Absorption heat pumps, as known in the art, have a series of heat exchangers for heat recovery 390 as a means of increasing the cooling Coefficient of Performance, including pre-cooling of the desorbed gas with heat recovery to preheat the strong solution prior to reaching the generator 380.

Another exemplary layout is shown in FIG. 5 that also discloses the multiple areas where heat transfer fluids through heat exchangers are in fluid communication between a biomass fuel conversion pretreatment process and an absorption heat pump system. The series of steps having heat transfer include: a) Biomass is combined with ionic liquid into a biomass solution 300 after being preheated by heat removed from the absorber of the absorption heat thermodynamic cycle 410 via heat recovery heat exchanger (heat sink 420); b) Pretreatment step including first stage of further raising the temperature and pressure of the solution by thermal means 370 and other catalytic or enzymatic processing as a means of transforming the biomass to a series of byproducts as known in the art for ultimate conversion to fuel; c) Biomass solution temperature is further raised by thermal means 360 into the generator/desorber 380; d) biomass pretreatment byproducts are isolated 60 from the desorbed ScCO2 utilizing means known in the art; e) heat recovery 390 from the desorbed ScCO2, which serves as precooling the ScCO2 subsequently transferring the thermal energy to the second stage of preheating the biomass solution within the pretreatment process 310; and the absorption thermodynamic cycle is completed through an expansion stage wherein the ScCO2 converts the thermal energy to mechanical or electrical energy via energy extraction device 70 as known in the art.

The further integration of solar concentration and flat panel as a thermal source with the supercritical biomass solution and a photocatalytic step, such as including Ciba® TINOLUX® BBS into a supercritical solar concentrator (or supercritical solar flat panel) yields a highly efficient and reactive photocatalytic system. The result is a hybrid photovoltaic system and biomass fuel, without being bound by theory, that achieves the high efficiency conversion of localized electrons to achieve oxidation degradation of biomass.

An exemplary ionic liquid for the inventive biomass conversion system is the use of the same ionic liquid utilized in the study of fruit ripening by high-resolution CNMR spectroscopy: ‘green’ solvents meet green bananas” by Diego A. Fort, Richard P. Swatloski, Patrick Moyna, Robin D. Rogers, and Guillermo Moyna, received (in Columbia, Mo., USA) 23 Oct. 2005, accepted 15 Dec. 2005, and first published as an Advance Article on the web 19 Jan. 2006 wherein banana pulps at any ripening stage were completely dissolved, which is in the IL 1-n-butyl-3-methylimidazolium chloride ([C4mim]Cl. ILs are capable of dissolving carbohydrates ranging from simple sugars to polysaccharides. Without being bound by theory, the nonhydrated chloride ions solvate carbohydrates by forming hydrogen bonds with their hydroxyl groups that in turn disrupt the complex intermolecular hydrogen bonding network present in many polysaccharides and promote their dissolution.

High Value Co-Products—Another embodiment is comprised of a means to alter the composition of the protein fraction within the biomass solution. Particularly the protein fraction is preferentially hydrolyzed into branched chain amino acids and peptides.

A particularly preferred pretreatment process occurs at temperatures where the protein fraction of the biomass solution is subjected to minimal denaturing. The utilization of enzymes to concurrently hydrolyze cellulose, hemicellulose, and lignincellulose with protein hydrolysis is a unique approach. The processing of proteins to protein hydrolysates, free amino acids, or peptides when combined with electron transfer mediators serves the dual role of debittering the resulting protein hydrolysates, free amino acids, or peptides after serving the role of enhancing the rate of hydrolysis during the pretreatment process. This dual role has the further advantage of not requiring extraction of the electron transfer mediator, when such electron transfer mediator is a food grade ingredient.

The specifically preferred method of processing a biomass solution is further comprised of debittering additives having both the ability to reduce the bitter taste of the free amino acids and peptides, and increasing the rate of at least one reaction selected from the group consisting of cellulose hydrolysis, protein hydrolysis, lignincellulose hydrolysis, electrochemical reduction of biomass conversion byproducts including carbon dioxide, electrochemical biodigestion, and electrochemical oxidation of biomass solution. Additional dual purpose additives (debittering and enhancing biomass conversion) additives include trehalose (provide thermal stability to enzymes and proteins), electron transfer mediators, electron donors including lactic acid, mineral ions selected from the group consisting of calcium, ferrous, cupric, manganous, and magnesium (enhancing electron transfer and impacting taste receptors). The biomass source is a feedstock selected from the group consisting of distiller's dried grain with solubles, corn, switchgrass, oat, and rice. Yet another embodiment is the isolation of protein fractions by enabling membrane filtration systems to effectively operate at pressures greater than the membrane design pressure as a means of increasing isolation efficiency. Thus the membrane filtration system is further comprised of a detector/controller to maintain the pressure across a microfiltration or nanofiltration membrane as a means of isolating protein fractions including protein hydrolysates, amino acids, and peptides wherein the pressure across the microfiltration or nanofiltration membrane is a pressure differential, and wherein the pressure differential is less than maximum microfiltration or nanofiltration membrane operating pressure.

1. A biomass solution comprised of a pretreatment solution, wherein the pretreatment solution comprises at least one working fluid selected from the group consisting of liquid ionic phosphates, polyammonium ionic liquid sulfonamides, and poly(ionic liquids), and combinations thereof.

2. The biomass solution according to claim 1 wherein the working fluid is further comprised of at least one gas selected from the group consisting of carbon dioxide, ammonia, and methane.

3. The biomass solution according to claim 1 further comprised of enzymes having at least hydrolyzing function selected from the group consisting of cellulose, hemicellulose, lignincellulose, and protein hydrolysis.

4. The biomass solution according to claim 3 wherein the enzymes are immobilized to the at least one working fluid.

5. The biomass solution according to claim 4 wherein the biomass solution is further comprised of microwave irradiation to increase the hydrolysis rate by a minimum of about 10% and wherein the biomass solution hydrolysis temperature is at least 5 degrees Fahrenheit lower than the pretreatment process void of microwave irradiation.

6. A biomass solution comprised of a pretreatment solution, wherein the pretreatment solution is comprised of an absorption heat pump having at least one working fluid component in fluid communication with the pretreatment process, wherein the at least one working fluid component is either the absorption heat pump refrigerant or refrigerant absorbent, and wherein the at least one working fluid increases the biomass surface area in the pretreatment process.

7. The biomass solution according to claim 6 wherein the absorption heat pump refrigerant removes moisture from the biomass solution.

8. A biomass solution comprised of at least one first working fluid A1 component from a biomass to biofuel conversion process in fluid communication with a biomass to biodiesel conversion process.

9. The biomass solution according to claim 8 wherein the at least one first working fluid A1 component is a byproduct of the biodiesel conversion process including glycerine or glycerol, and decreases the biomass moisture content of the biomass to biofuel conversion process.

10. The biomass solution according to claim 9, further comprised of a regeneration process to remove moisture from the at least one first working fluid A1 component, and wherein the regeneration process utilizes recovered waste heat from an at least one second working fluid A2 component in fluid communication with both biomass to biofuel conversion process and biomass to biodiesel conversion processes.

11. The biomass solution according to claim 8, further comprised of a power generation cycle to produce electricity utilized for at least one function selected from the group consisting of microwave irradiation, electrochemical reduction, and electrolysis.

12. The biomass solution according to claim 8, further comprised of an electrochemical reduction process to convert carbon dioxide byproduct of the biomass to biofuel conversion process into an input for the biomass to biofuel conversion process.

13. The biomass solution according to claim 8, further comprised of carbonate solvents as a means of increasing the electrical conductivity and decreasing the at least one working fluid viscosity.

14. The biomass solution according to claim 12 wherein the carbon dioxide byproduct is absorbed by at least one working fluid A1.

15. The biomass solution according to claim 14 wherein the carbon dioxide byproduct absorbed by the at least one working fluid A1 is further processed by means including reactions of polymerizing carbon dioxide, carbonate synthesis, or electrochemical reduction to methane.

16. A biomass solution comprised of a pretreatment solution, wherein the pretreatment solution is comprised of at least one step selected from the group consisting of electrochemical, electrolysis, electrocatalytic, and photocatalytic process step, and wherein the pretreatment solution is comprised of at least one working fluid additive selected from the group combination of nanoscale conductors and semi-conductors as a means of increasing quantum mean free path, ionic liquids, liquid ionic phosphates polyammonium ionic liquid sulfonamides, quantum dots, copper, Fe2+ ions, iron-sulfur cluster, or electrides.

17. The biomass solution according to claim 16, further comprised of a process step to remove sources of electron donors prior to hydrolysis including lignin, antioxidants, polyphenols, and aromatic compounds.

18. The biomass solution according to claim 16, further comprised of at least one working fluid additive selected from the group consisting of electron transfer mediator including iron salts, derivatives of iron salts, potassium salts, lactic acid salts, derivatives of potassium salts, derivatives of lactic acid salts, phytic acid, gallic acid, potassium ferricyanide, polyoxometalates, violuric acid, polycationic protein, thialoto-bridged complexes, thiolated complexes, metalloproteins, protein complexes having an iron-sulfur cluster, trehalose complexes, iron-sulfur cluster, sodium-ammonia, sulfur-ammonia, a chitosan complex including chitosan lactate, chitosan alpha lipoic acid, and thiolated chitosan, nanoscale catalyst, electrocatalyst, photocatalyst, electron donor, electron acceptor, ultraviolet absorber, infrared absorber, quantum dot, nanoscale powder, enhancing electron transfer including iron salts, derivatives of iron salts, potassium salts, lactic acid salts, derivatives of potassium salts, derivatives of lactic acid salts, phytic acid, gallic acid and combinations thereof.

19. The biomass solution according to claim 16, further comprised of a control system with non-linear algorithms capable of determining the maximum operating revenue in real-time by monitoring at least one parameter selected from the group consisting of cost and price of electricity, cooling cost and price per btu, heating cost and price per btu, carbon dioxide emission credits, cost and price of methanol per btu, cost and price of resulting biofuels per btu, conversion factor of electricity for electrochemical reactions, and operating parameters of a reverse fuel cell for electrochemical reduction of at least one component of the biomass solution.

20. A biomass solution comprised of a hydrolysis process, wherein the hydrolysis process is terminated within a rapid expansion step further contained within an energy extraction device including gerotor, pressure exchanger, and quasiturbine, or a microchannel device having channels less than 10 microns as a means of reducing precipitated cellulose.

21. The biomass solution according to claim 20 wherein the rapid expansion step occurs in a series of independent pressure drop stages comprised of at least a first pressure drop stage and a second pressure drop stage.

22. The biomass solution according to claim 21, further comprised of at least one working fluid having a pressure greater than the at least one working fluid's supercritical pressure.

23. The biomass solution according to claim 21 wherein the first pressure drop stage has a pressure below at least one working fluid's supercritical pressure.

24. The biomass solution according to claim 21 wherein the biomass solution is infused with at least one working fluid additive selected from the group consisting of monomers, polymers solubilized in the at least one working fluid, microspheres, and nanoscale powders having particle size less than 100 nanometers.

25. The biomass solution according to claim 21 wherein the biomass solution is mixed by at least one process intensification mixer including hydrodynamic cavitation devices, spinning disk, or spinning tube in tube.

26. The biomass solution according to claim 24 wherein the microspheres are further comprised of immobilized enzymes, immobilized catalysts, or combinations thereof.

27. The biomass solution according to claim 24 wherein the working fluid additives are further processed into polymers, copolymers, or block copolymers.

28. The biomass solution according to claim 21 wherein the second pressure drop stage occurs within a pressure exchanger wherein the high pressure fluid is the biomass solution from the exit of the pretreatment process and wherein the low pressure fluid is the biomass solution prior to the pretreatment process.

29. A biomass solution comprised of a rapid expansion pretreatment process having an expanded gas and a power generating thermodynamic cycle wherein the expanded gas from the pretreatment process is in fluid communication with a condenser of the power generating thermodynamic cycle as a means of increasing the thermodynamic cycle efficiency of both the biomass pretreatment process and the power generating thermodynamic cycle.

30. The biomass solution according to claim 29 further comprised of a waste heat recovery device to recover thermal energy from the condenser of the power generating thermodynamic cycle wherein the thermal energy is further increased by the heat of absorption in the subsequent mixing of the expanded gas into at least one absorbent prior to recombining with the biomass solution.

31. The biomass solution according to claim 30 wherein the heat of absorption is in fluid communication with the power generating thermodynamic cycle as a preheat stage.

32. The biomass solution according to claim 29 further comprised of a waste heat recovery process step wherein the waste heat is utilized for at least one function selected from the group consisting of preheating the inputs of the rapid expansion pretreatment process, thermal hydraulic pump, and thermal inputs of an absorption heat pump as a means of increasing fluid pressure.

33. The biomass solution according to claim 29 further comprised of a waste heat recovery device to recover thermal energy from the condenser of the power generating thermodynamic cycle in fluid communication with the biomass solution and wherein the thermal energy is utilized as at least a partial thermal energy source for an endothermic reaction.

34. The biomass solution according to claim 29 wherein the power generating thermodynamic cycle is comprised of a working fluid having at least a first working fluid W1 and a second working fluid W2.

35. The biomass solution according to claim 34 wherein the first working fluid W1 and the second working fluid W2 are each individually selected from the group consisting of carbon dioxide, ammonia, methanol, ethanol, butanol, and water.

36. The biomass solution according to claim 29 wherein the power generating thermodynamic cycle is selected from the group consisting of binary Organic Rankine, Goswami, Kalina, and Carnot cycles.

37. A biomass solution comprised of a protein fraction, wherein the protein fraction is preferentially hydrolyzed into branched chain amino acids and peptides.

38. The biomass solution according to claim 37 wherein the protein fraction hydrolyzed into branched chain amino acids and peptides is further comprised of debittering additives having both the ability to reduce the bitter taste of the free amino acids and peptides, and increase the rate of at least one reaction selected from the group consisting of cellulose hydrolysis, protein hydrolysis, lignincellulose hydrolysis, electrochemical reduction of biomass conversion byproducts including carbon dioxide, electrochemical biodigestion, and electrochemical oxidation of biomass solution.

39. The biomass solution according to claim 38 wherein the debittering additives include trehalose, electron transfer mediators, electron donors including lactic acid, mineral ions selected from the group consisting of calcium, ferrous, cupric, manganous, and magnesium.

40. The biomass solution according to claim 37 wherein the biomass solution is a feedstock selected from the group consisting of distiller's dried grain with solubles, corn, switchgrass, oat, and rice.

41. The biomass solution according to claim 37, further comprised of a detector/controller to maintain the pressure across a microfiltration or nanofiltration membrane as a means of isolating protein fractions including protein hydrolysates, amino acids, and peptides, wherein the pressure across the microfiltration or nanofiltration membrane is a pressure differential, and wherein the pressure differential is less than the maximum microfiltration or nanofiltration membrane operating pressure.
